(19) United States
(12) Patent Application Publication
(10) Pub. No.: US 2013/0177506 A1
(43) Pub. Date: Jul. 11, 2013

(54) Bio-Available Chlorogenic Acid Preparations for Supplemental Human Consumption and Use

(71) Applicants: Parker E. Atkins, Boerne, TX (US); Donald J. Wold, Boerne, TX (US)

(72) Inventors: Parker E. Atkins, Boerne, TX (US); Donald J. Wold, Boerne, TX (US)

(21) Appl. No.: 13/663,397

(22) Filed: Oct. 29, 2012

Related U.S. Application Data
(60) Provisional application No. 61/551,979, filed on Oct. 27, 2011.
(60) Provisional application No. 61/602,639, filed on Feb. 24, 2012.

(51) Int. Cl. A61K 31/216; A23L 1/30; A61K 9/68

(52) U.S. Cl. CPC A61K 31/216; A61K 9/0058; A23L 1/3002
USPC 424/48; 560/75; 514/533

(57) ABSTRACT

A chewing gum, creamer, powdered supplement or other confection, as a food supplement formulated to combine natural or artificial sweeteners and flavorings with CGA, packaged for retail sale to consumers, for providing an ideal bio-available delivery mode for CGA, a recognized antioxidant and natural metabolic stimulant, as a ready supplement for bioactive immune support and to aid users in management of weight loss, obesity and/or glucose management, or for general health and well-being of users.

### NON-PROVISIONAL APPLICATION

This application is a non-provisional application filed under 37 CFR 1.53(b).

### CLAIM OF PRIORITY TO PRIOR APPLICATION

The present application claims the benefit of prior filed U.S. Provisional Application, Ser. No. 61/551,979, filed Oct. 27, 2011 and U.S. Provisional Application Ser. No. 61/602,639, filed Feb. 24, 2012. By this reference, the full disclosure of U.S. Provisional Application Ser. Nos. 61/551,979 and 61/602,639 are incorporated herein as though now set forth in their entirety.

### FIELD OF THE INVENTION

The present invention relates to the field of promoting human health and well being, particularly through phytochemical polyphenol preparations and, more particularly, through phytochemical polyphenol preparations of chlorogenic acid (“CGA”) naturally found in coffee beans and coffee bean extracts.

### BACKGROUND

Obesity, insulin resistance, and type 2 Diabetes are closely associated with chronic inflammation characterized by abnormal cytokine production, increased acute-phase reactants and other mediators, and activation of a network of inflammatory signaling pathways. Chronic disturbance of metabolic homeostasis, such as occurs in malnutrition or over-nutrition, can lead to aberrant immune responses. Counteracting and/or preventing such conditions have long been an important public need in order to promote health and well being.

Phytochemicals are natural biologically-active chemical compounds derived from plants, and polyphenols are one of the most widely studied classes of phytochemicals. While polyphenol phytochemicals are important intermediaries that influence the metabolism and serve various antimicrobial and other defensive purposes for the plants in which they occur naturally, they have also been used as functional foods and as drugs since ancient times, and their physiochemical properties have long made them the central focus of modern botanical, nutraceutical, and health food industries.

Chlorogenic acid (CGA, which is actually a family of acid compounds) was clearly identified and described as far back as 1846 and is one of the roughly 10,000 different types of known phytochemicals. Commonly characterized as esters formed between quinic acid and caffeic acid (or another cinnamic or hydroxycinnamic acid), CGA can also be characterized as a hydroxycinnamic acid glycoside. Today, CGA is generally recognized as safe (GRAS) for human consumption by the U.S. Food and Drug Administration. CGA generally falls under CAS Registry Number 327-97-9. Its major isomers are caffeoylquinic, feruloylquinic and dicaffeoylquinic acids (CQA, FQA and diCQA, respectively), with numerous variations recognized within such isomer groupings, such as 3-, 4- or 5-CQA; 3- or 4-FQA; and 3,4-, 3,5-, or 4,5-diCQA. In addition to its primary metabolites of caffeic, cinnamic and quinic acids, its recognized secondary metabolites also include benzoic acid, hippuric acid, carboxylic acid, 3,4-dihydroxyphenylpropionic acid, 3-hydroxyphenylpropionic acid, 3-hydroxybenzoic acid, 3-hydroxyhippuric acid, ferulic acid, isoferulic acid, and m-coumaric acid.

Hydroxycinnamic acids comprise a class of polyphenols found in many plants and food sources for both animals and humans, but especially fruits such as apples, pears, peaches, plums and cherries. Hydroxycinnamic acids such as CGA are well known polyphenol phytochemicals with health benefits that have long been widely known and studied. They are used biochemically in metabolic signaling processes and are widely recognized as providing foods with antioxidant, anti-inflammatory, anticarcinogenic, antiaging and antimicrobial properties in naturally functional foods. In the antioxidant role, their physicochemical properties help prevent oxidation by chelating metals and scavenging oxygen-free radicals (or reactive oxygen species).

Epidemiological studies have suggested that consumption of phytonutrients from black tea and coffee may have significant health benefits. CGA, particularly, is generally produced in nature from esterification of caffeic acid and is found in especially high concentration in certain coffee bean and black tea varietals, as well as in bamboo, blueberries and tomatoes. Daily intake of CGA by coffee drinkers is 0.5-1 g/d while non-drinkers ingest <100 mg/d. CGA compounds are thought to be beneficial due to their antioxidant and anti-inflammatory characteristics that help to: reduce the risk of atherosclerosis and cardiovascular disease; reduce risk of hypertension; lower blood levels of LDL cholesterol; attenuate intestinal glucose absorption rates; lower the risk of type 2 diabetes; inhibit human hepatocellular carcinoma cell-line proliferation; inhibit synthesis of the pro-inflammatory cytokine tumor necrosis factor-alpha (TNF-α is the first identified link between inflammation and obesity); inhibit allergic rhinitis; protect against oxidative damage and inhibit potential mutagenic and carcinogenic reactions; dampen levels of reactive oxygen species (ROS) in antigen-IgE-activated mast cells; modulate histamine release; lower the risk of prostate cancer; reduce the risk of pancreatic cancer; inhibit spasmodic activity; increase serum adiponectin concentrations; promote weight loss, etc.”

Obesity is associated with an array of additional heath problems, including increased risk of insulin resistance, Type 2 diabetes, fatty liver disease, atherosclerosis, degenerative disorders including dementia, airway disease and some cancers” (Hotamisligil, Nature 444/14: 2006). The potent radical scavenging attributes of CGA and related compounds contribute to the efficient operation of the immune system. The immune system and metabolic regulation are tightly integrated, working together to maintain homeostasis essential to wellness, while the link between them is a delicate balance. Although there are short-term compensatory and adaptive measures to keep this delicate balance in check, the outcome is often detrimental when one arm overwhelms the other in the long term.

There are no known adverse effects linked to normal consumption of CGA. However, ultra-high doses of CGA (2 g/d equivalent to the load of CGA in 1.5 L/d strong coffee) are at least partly responsible for increased levels of homocysteine in postprandial plasma. This effect may be mediated by ingestion of vitamin B-6, vitamin B-12 and folic acid or folate (forms of water-soluble vitamin B-9).

Green coffee beans have the highest percentage of CGA per dry weight found in plants (6-12%). Nine major and fourteen minor CGA compounds have been identified in green coffee extract (Farah et al., Journal of Nutrition, 138: 2008), a primary source of CGA for the preferred method herein defined. However, the amount of CGA in prepared coffee depends on multiple variables among which are place of origin, time of harvest, grinding and especially roasting. Decline in the amount of CGA exhibited in coffee is directly proportional to increased roasting time and temperature. Sustained roasting may result in almost total loss of CGA, thus forfeiting its many potential health benefits. Contrariwise, CGA lactones formed during roasting have been shown to have positive effects on brain function. Unfortunately, significant amounts of the phytochemicals present in freshly harvested plants are destroyed or removed in the process of preparing the plant for normal use, such that industrially processed plants likely contain far fewer and may thus be less beneficial than unprocessed foods. To compound the problems, attempts to refortify plant products after processing, such as by reintroduce the phytochemicals to the processed plant, commonly results in a preparation with bioavailability that is less than optimum.

### SUMMARY AND DETAILED DESCRIPTION OF PREFERRED EMBODIMENTS

The present invention provides a packaged preparation and related methods for managing weight loss, glucose levels and obesity, and/or for otherwise enhancing a person's health and general well-being. Preferred embodiments provide as much through the provision of food products with polyphenol additives and/or through the addition of polyphenol food additives to their food products prior to consumption. Presently preferred methods involve provision of chlorogenic acid (CGA) nutritional supplements in one or more of the various preferred delivery forms of the present invention. Certain particularly preferred delivery forms that incorporate additional aspects of innovation include the provision of CGA supplemental dosings in chewing gums commercialized through consumer retail outlets.

Although the invention should be understood first in the context of the final claims of this and/or any dependent patent applications, other preferred delivery forms of certain aspects of the invention may include confections, coffee additives, lozenges, nasal sprays or any other acceptable method of delivery whatsoever whether enteral or parenteral (intravenous, subcutaneous or intramuscular).

More particular embodiments of certain aspects of the invention provide as much through the use of single dose supplement packets of powdered polyphenol-containing food additives that can be and preferably are made available at customer-accessible locations of common beverage preparation and/or consumption. Such locations include table-top locations in restaurants, counter-top locations in convenience stores, and condiment stations in coffee shops, as well as other analogous location. Through availability at such locations, consumers are encouraged and able to mix the supplements into their coffee or other beverage of choice, for the management of obesity and other metabolic disorders.

Preferably, the supplements are provided in single-serving packets that are made available in numbers at or near the point of food or beverage consumption, such as in a cup or other conventional receptacle for disposable single-use packets like sugar, sweetener or other coffee condiment single-serving packages. As would be understood by those in the food industry, the individual packets would be marked, labeled or otherwise adequately identified to clearly distinguish the unique character of the polyphenol-packets in contrast to conventional condiment packets. One of the results is a more convenient and enjoyable system for managing obesity risks, as compared to the taking of pills or the like.

The preferred formulations and recommended daily consumption amounts of the CGA nutritional supplements are predicated on the ingestion of approximately 1 g/d CGA by all means whatsoever, including drinking coffee, ingesting food containing CGA, and consuming the CGA nutritional supplements encompassed within the teachings of this invention. The described CGA nutritional supplements are intended to provide a means for non-coffee drinkers to increase their daily intake of CGA; to increase the percentage of CGA in the coffee cup; to replace CGA lost in the process of preparing coffee; to provide vehicles for ingesting CGA and its metabolites when drinking coffee is not convenient; to promote antioxidant activities in support of the metabolic system; and to enhance anti-inflammatory activities in counteracting metabolic disorders while supporting homeostasis of the immune system in its battle against non-self and autoimmune deficiencies.

A particularly preferred embodiment is in the form of a powdered preparation of chlorogenic acid (CGA), combined with other consumable substances in granular or powder form, with the combination packaged in single-use packets much like single-use sugar and sweetener packets. Such preparations are preferably prepared by measured combinations of powdered CGA (available from various sources) together with sweeteners and/or other food additive powders. The particular quantities may vary, although one preferred embodiment uses proportions as in “Formulation Example” described below, excluding the gum base components.

In a preferred class of preferred embodiments, the other consumable substances that are combined with the CGA include one or more sweeteners that are acceptable for use as a coffee sweetener. While some embodiments may use sugar as the sweetener, preferred embodiments use a low-calorie or no-calorie sweetener, preferably in granular or powder form. Other embodiments may use any legally commercialized sweetener, such as any one or more of Xylitol, Sucralose or any of the following naturally derived sugar substitutes and artificial sugar substitutes.

In addition to the use of natural sugar as a substitute, there are naturally derived sugar substitutes and artificial sugar substitutes; any approved substances from these lists, either alone or in combination may be contemplated for use as follows:

List of Potential Natural Sugar Substitutes:

Brazzein

Curculin

Erythritol

Glycerol

Glycyrrhizin

Hydrogenated starch hydrolysates

Inulin

Isomalt

Lactitol

Luo han guo

Mabinlin

Maltitol

Malto-oligosaccharide

Mannitol

Miraculin

Monatin

Monellin

Osladin

Pentadin

Sorbitol

Stevia

Tagatose

Thaumatin

List of Potential Artificial Sugar Substitutes:

Acesulfame potassium

Alitame

Aspartame

Salt of aspartame-acesulfame

Glucin

Neohesperidin dihydrochalcone

Neotame

Saccharin

Another alternative, particularly preferred class of embodiments provides polyphenol supplements in the form of a coffee creamer preparation of chlorogenic acid (CGA) dissolved or dispersed in a liquid creamer for coffee and the like. Preferably, the polyphenol-creamer combinations are packaged in otherwise conventional single-use coffee creamer packages that contain a liquid creamer (either dairy or non-dairy), although labeling would be adapted to clearly identify the unique character of the polyphenol-creamer in contrast to conventional creamers.

In additional to CGA, supplement preparations may also include other food additives such as other phytochemicals, vitamins, minerals, amino acids, and dietary substances, and many will preferably be made available in various flavors. Flavor variations preferably include chocolate, vanilla, cappuccino, hazelnut, and cinnamon, with or without sweeteners.

Other preferred embodiments involve using such preparations as nutritional supplements in combination with coffee or other beverages or other food products. Still other embodiments involve the combination of such preparations in various forms to produce chewing gums, confections, coffee additives, lozenges, nasal sprays or any other acceptable method of delivery whatsoever whether enteral or parenteral (intravenous, subcutaneous or intramuscular). The purpose of the preferred method formulation in any or all of its delivery incarnations is to promote bioavailability and absorption of CGA and its metabolites primarily but not exclusively throughout the alimentary canal whether in the oral cavity, stomach, small intestine or colon. Moreover, the preferred method covers a host of polyphenolic antioxidants and/or anti-inflammatory agents when synergistically combined and/or formulated with said CGA nutritional supplements in immune and metabolic functions including but not limited to specifically quercetin; rutin (quercetin-3-rutinoside); ellagic acid; kaempferol; myricetin; RCM-101; and generally phlorotannins; catechins; anthocyanins; and the class of flavonoids encompassing flavonols; flavones; flavonones; anthocyanidins and isoflavones; as well as certain stilbenoids not exclusively but especially resveratrol; and any and all phytocehmicals when specifically tied to the described CGA preferred method.

The following formulation examples illustrate additional alternative embodiments for a packaged food product embodiment of one of the preferred forms:

### FORMULATION EXAMPLE

Product: CGA Nutritional Food Supplement
Preferred Product Color: Tan to Brown
Preferred Single-Dose Package Color: Tan or Green hue
Preferred Serving Size: 1
Weight per unit (mg): 2700 mg with Gum Base;
   553 mg without Gum Base

| Ingredient | | mg/ | mg/ | Total | |
|---|---|---|---|---|---|
| # | Potency | unit | serving | mg | Ingredient Description |
| 1 | 1 | 2147 | 2147 | 2147 | Gum Base (omitted in some |
| | | | | | embodiments) |
| 2 | 1 | 20 | 20 | 20 | Caffeine |
| 3 | 1 | 300 | 300 | 300 | 45% Chlorogenic acid |
| | | | | | extract |
| 4 | 1 | 1 | 1 | 1 | Citric Acid (1) |
| 5 | 1 | 25 | 25 | 25 | Cappucino Flavor |
| 6 | 1 | 100 | 100 | 100 | Xylitol Direct |
| | | | | | Compressable (granular) |
| 7 | 1 | 7 | 7 | 7 | Sucralose |
| 8 | 1 | 100 | 100 | 100 | Non Fat Dry Milk Powder |

### Preferred Ingredient Dosage Ranges

| | | Dosage | |
|---|---|---|---|
| Ingredient # | Ingredient Description | Range | Units |
| 1 | Gum Base | 0.0-3000 | mg |
| 2 | Caffeine | 0.0-100 | mg |
| 3 | 45% Chlorogenic | 100-1000 | mg (i.e., compares |
| | acid extract* | | to 45-450 mg |
| | | | 100% CGA) |
| 4 | Citric Acid (1) | 0.0-2.0 | mg |
| 5 | Cappucino Flavor | 15-100 | mg |
| 6 | Xylitol Direct | 0.0-200 | mg |
| | Compressable (granular) | | |
| 7 | Sucralose | 0.0-200 | mg |
| 8 | Non Fat Dry Milk | 0.0-200 | mg |
| | Powder | | |

*Chlorogenic Acid (CGA) may vary in concentration from different suppliers. One preferred method and formulation uses a CGA at 45% purity, which results in an actual concentration of 45% * 300 mg = 135 mg CGA per unit. A supplier with a product containing 75% CGA would result in 180 mg of the CGA extract to yield the same dose of 135 mg per unit. Other polyphenol alternatives may be considered to the extent consistent with the overall disclosure in light of the claim language and its course of prosecution.

Certain preferred embodiments provide a CGA supplement delivered as a chewing gum and packaged as a consumer chewing gum product, preferably available through consumer retail outlets. Although alternative forms may fall within the scope of the invention, the preferred chewing gum form is embodied in the Chiclet-type form with a slightly harder yet chewable shell surrounding the primary chewing gum confection. Such formulations preferably include: (1) a gum base; (2) a CGA component; (3) caffeine (preferably); (4) natural or artificial sweeteners (preferably Xylitol or Sucralose); and (5) flavorings.

Such chewing gum embodiments preferably provide an innovative chewing gum delivery mode for CGA and its benefits, all in a great tasting product that will tend to remain bio-available for extended periods in a user's mouth so that it can more readily be absorbed sub-lengually. As a result, such embodiments provide ideal delivery of a recognized antioxidant and natural metabolic stimulant, for bioactive immune support. Such embodiments also provide delivery of other polyphenol compounds that are naturally found in coffee but are commonly removed, converted or reduced through typical coffee roasting.

The following provides more descriptive information for some of the preferred chewing gum embodiments. Particularly, a packaged sugar-free variation is preferred, which packages twenty pieces of chewing gum in a box with labeling for consumer purchase, with each piece of gum being a single serving and each serving approximating the CGA of one cup of coffee, according to the following information:

CGA Nutritional Food Supplement in Chewing Gum form

Product Color: Tan to Brown

Possible Labeling Information:

Supplement Facts

Serving Size: 1 Piece (2.5 g)

Serving Per Container: 20

Amount Per Serving % Daily Value*

Calories 5

Total Fat 0 g 0%

Sodium 0 mg 0%

Total Carbohydrate 2 g 1%

Protein 0 g

Green Coffee extract** 100 mg †
Caffeine 60 mg †

† Daily Value (DV) not established.

**Chlorogenic Acid Content=60 mg in Extract per serving.

Not a significant source of calories from fat, saturated fat, cholesterol, dietary fiber, sugars, vitamin A, vitamin C, calcium, and iron.

*Percent Daily Values are based on a 2,000 calorie diet.
Other Ingredients: Maltitol, Gum Base, Sorbitol, Xylitol, Maltitol Syrup, Green Coffee Extract (Containing Chlorogenic Acid), Caffeine, Gum Arabic, Natural and Artificial Flavor, Glycerine, Titanium Dioxide (color), Sucralose, Resinous Glaze, Soy Lecithin, Carnauba Wax, and Neotame.

### Dosage Range and Known Equivalents

| Ingredient | | | | Known |
|---|---|---|---|---|
| # | Ingredient Description | Dosage Range | Units | Equivalents |
| 1 | Gum Base | 0.0-3000 | mg | None |
| 2 | Caffeine | 0.0-100 | mg | None |
| 3 | 45% Chlorogenic | 200-1000 | mg | * |
| | acid extract | | | |
| 4 | Citric Acid (1) | 0.0-2.0 | mg | None |
| 5 | Cappucino Flavor | 15-100 | mg | None |
| 6 | Xylitol Direct | 0.0-200 | mg | None |
| | Compressable | | | |
| | (granular) | | | |
| 7 | Sucralose | 0.0-200 | mg | None |
| 8 | Non Fat Dry Milk | 0.0-200 | mg | None |
| | Powder | | | |

The gum base of various chewing gum embodiments may include any of the following ingredients or substitute ingredients, as follows:

| Family | Genus and species |
|---|---|
| Sapotaceae: | |
| Chicle | Manilkara zapotilla Gilly and Manilkara chicle Gilly. |
| Chiquibul | Manilkara zapotilla Gilly. |
| Crown gum | Manilkara zapotilla Gilly and Manilkara chicle Gilly. |
| Gutta hang kang | Palaquium leiocarpum Boerl. and Palaquium oblongifolium Burck. |
| Massaranduba balata | Manilkara huberi (Ducke) Chevalier. |
| (and the solvent-free | |
| resin extract of | |
| Massaranduba balata) | |
| Massaranduba chocolate | Manilkara solimoesensis Gilly. |
| Nispero | Manilkara zapotilla Gilly and Manilkara chicle Gilly. |
| Rosidinha (rosadinha) | Micropholis (also known as Sideroxylon) spp. |
| Venezuelan chicle | Manilkara williamsii Standley and related spp. |
| Apocynaceae: | |
| Jelutong | Dyera costulata Hook, F. and Dyera lowii Hook, F. |
| Leche caspi (sorva) | Couma macrocarpa Barb. Rodr. |
| Pendare | Couma macrocarpa Barb. Rodr. and Couma utilis (Mart.) Muell. Arg. |
| Perillo | Couma macrocarpa Barb. Rodr. and Couma utilis (Mart.) Muell. Arg. |
| Moraceae: | |
| Leche de vaca | Brosimum utile (H.B.K.) Pittier and Poulsenia spp.; also Lacmellea |
| | standleyi (Woodson), Monachino (Apocynaceae). |
| Niger gutta | Ficus platyphylla Del. |
| Tunu (tuno) | Castilla fallax Cook. |
| Euphorbiaceae: | |
| Chilte | Cnidoscolus (also known as Jatropha) elasticus Lundell and Cnidoscolus |
| | tepiquensis (Cost. and Gall.) McVaugh. |
| Natural rubber (smoked | Hevea brasiliensis. |
| sheet and latex solids) | |

Synthetic Specifications

| | |
|---|---|
| Butadiene-styrene rubber | Basic polymer. |
| Isobutylene-isoprene | |
| copolymer (butyl rubber) | |
| Paraffin | Synthesized by Fischer-Tropsch process from carbon monoxide and |
| | hydrogen which are catalytically converted to a mixture of paraffin |
| | hydrocarbon. Lower molecular weight fractions are removed by |
| | distillation. The residue is hydrogenated and further treated by percolation |
| | through activated charcoal. The product has a congealing point of 93deg.-99 |
| | deg. C as determined by ASTM method D938-71 (Reapproved 1981), |
| | “Standard Test Method for Congealing Point of Petroleum Waxes, |
| | Including Petrolatum,” a maximum oil content of 0.5 percent as |
| | determined by ASTM method D721-56T, “Tentative Method of Test for Oil |
| | Content of Petroleum Waxes,” and an absorptivity of less than 0.01 at |
| | 290 millimicrons in decahydronaphthalene at 88 deg. C. as determined by |
| | ASTM method D2008-80, “Standard Test Method for Ultraviolet |
| | Absorbance and Absorptivity of Petroleum Products,” which are |
| | incorporated by reference. |
| Petroleum wax | Complying with 172.886. |
| Petroleum wax synthetic | Complying with 172.888. |
| Polyethylene | Molecular weight 2,000-21,000. |
| Polyisobutylene | Minimum molecular weight 37,000 (Flory). |
| Polyvinyl acetate | Molecular weight, minimum 2,000. |

Plasticizing Materials (Softeners)

| | |
|---|---|
| Glycerol ester of partially | Having an acid number of 3-8, a minimum drop-softening point of 109 |
| dimerized rosin | deg. C., and a color of M or paler. |
| Glycerol ester of partially | Having an acid number of 3-10, a minimum drop-softening point of 79 |
| hydrogenated gum or wood | deg. C., and a color of N or paler. |
| rosin | |
| Glycerol ester of polymerized | Having an acid number of 3-12, a minimum melting-point of 80 deg. C., |
| rosin | and a color of M or paler. |
| Glycerol ester of gum rosin | Having an acid number of 5-9, a minimum drop-softening point of 88 deg. |
| | C., and a color of N or paler. The ester is purified by steam stripping. |
| Glycerol ester of tall oil rosin | Having an acid number of 2-12, a softening point (ring and ball) of 80 deg.-88 |
| | deg. C., and a color of N or paler. The ester is purified by steam |
| | stripping. |
| Glycerol ester of wood rosin | Having an acid number of 3-9, a drop-softening point of 88 deg. C.-96 |
| | deg. C., and a color of N or paler. The ester is purified by steam stripping. |
| Lanolin | |
| Methyl ester of rosin, partially | Having an acid number of 4-8, a refractive index of 1.5170-1.5205 at 20 |
| hydrogenated | deg. C., and a viscosity of 23-66 poises at 25 deg. C.. The ester is purified |
| | by steam stripping. |
| Pentaerythritol ester of | Having an acid number of 7-18, a minimum drop-softening point of 102 |
| partially hydrogenated gum or | deg. C., and a color of K or paler. |
| wood rosin | |
| Pentaerythritol ester of gum or | Having an acid number of 6-16, a minimum drop-softening point of 109 |
| wood rosin | deg. C., and a color of M or paler. |
| Rice bran wax | Complying with 172.890. |
| Stearic acid | Complying with 172.860. |
| Sodium and potassium | Complying with 172.863. |
| stearates | |

Terpene Resins

| | |
|---|---|
| Synthetic resin | Consisting of polymers of [alpha]pinene, [beta]pinene, and/or dipentene; |
| | acid value less than 5, saponification number less than 5, and color less |
| | than 4 on the Gardner scale as measured in 50 percent mineral spirit |
| | solution. |
| Natural resin | Consisting of polymers of [alpha]-pinene; softening point minimum 155 |
| | deg. C., determined by U.S.P. closed-capillary method, United States |
| | Pharmacopeia XX (1980) (page 961). |

Miscellaneous

Sodium sulfate
Sodium sulfide

### Ingredient Substitutes in the Preferred Formulation and Method

Some preferred alternative formulations and methods may include the use of substitute ingredients or an original ingredient with synergistic ingredients that result in similar or superior health benefits as those previously discussed. The primary ingredient is followed by substitute and/or synergistic ingredients in the preferred method as follows:

I. Caffeine Ingredient
No substitute ingredients are noted, although alternative embodiments provide formulations without any significant caffeine, and other alternatives provide formulations to achieve caffeine ingredients of less than 10 mg, or less than 15 mg, caffeine per packet/dose/unit.

II. Chlorogenic Acid (CGA)
This application includes polyphenolic antioxidants and/or anti-inflammatory agents when synergistically combined and/or formulated with said CGA nutritional supplement including but not limited to: quercetin; rutin (quercetin-3-rutinoside); ellagic acid; kaempferol; myricetin; RCM-101; and generally phlorotannins; catechins; anthocyanins; and the class of flavonoids encompassing flavonols; flavones; flavonones; anthocyanidins and isoflavones; as well as certain stilbenoids not exclusively but especially resveratrol; and any and all phytochemicals when specifically combined with the described CGA nutritional supplement.

III. Citric Acid
Any approved natural preservative that does not modify the dose or bioavailability of the nutritional supplement.

IV. Cappuccino Flavor
Substitutes may include any commercially available natural and artificial flavors.

V. Non-Fat Dry Powdered Milk (for Some but Not All Preferred Embodiments)
No substitute ingredients noted.

For additional perspective on the invention, it should be understood that non-absorbed polyphenols reach the colon. In the colon, the microbiota (e.g.; Escherichia coli, Bifidobacterium sp., Lactobacillus sp., Bacteroides sp., Eubacterium sp) hydrolyses [sic] the glycosides to aglycones, which can further be metabolized to aromatic acids like phenylacetic, phenylpropionic, phenylvaleric and benzoic acid” (Bosscher et al., Journal of Physiology and Pharmacology, 60: 2009). Therefore, said preferred method encompasses microbiota in the colon when such microbiota can be specifically tied to the actions of the herein described CGA nutritional supplements. Before entering the bloodstream, polyphenols are structurally modified in a conjugation process for the most part in the liver. All CGA compounds, while variously absorbed and metabolized, are bioavailable in humans. The polyphenols are extensively modified during the absorption: the glycosides could be hydrolyzed in the small intestine or in the colon, and the released aglycones could be absorbed. (D'Archivio et al., International Journal of Molecular Sciences 11: 2010).

For purposes of these descriptions, except to the extent limited by the prior art or to the extent clearly, expressly and unequivocally limited in a particular context, it should be understood that reference to any compound herein should be construed as broadly as possible. Accordingly, even if the molecular formula and structure for a compound or group of compounds may be generally expressed in limited ways, it should be assumed that reference to such compound or group broadly encompasses any compound that substantially fulfils at least one of the corresponding definitions that may be used for the compound or group in the public domain. For instance, this description generally refers to CGA as a specific compound even though those of skill in the art would recognize that CGA may have variations in its formula or structure, all of which should be understood as falling within the scope of CGA. Likewise, with any compound referenced herein, unless Applicant expressly and unequivocally clarifies that the reference should be more limited in a particular context, all references to that compound should be presumed to read as broadly as possible for purposes of construing the scope of the invention claimed.

In addition, to the extent permitted by the prior art and prosecution, any reference in the claims or elsewhere to a particular compound or group should also be understood as encompassing equivalents, including equivalents that would accomplish substantially the same function as the referenced compound or group. To that same extent, i.e., so long as there is not a requirement for a more limited interpretation in the prosecution history and/or based on an otherwise invalidating prior art reference, such equivalents should be presumed to include all homologues, isomers, lactones, metabolites, derivatives, fragments, variants, analogs, substitutes, alternatives and the like.

Presently preferred embodiments of the present invention are in the form of a supplement that is added to food products, preferably beverages, for delivery of CGA through sublingual and other mucosal membranes as well as through the gastro intestinal tract. In such embodiments, the formula as described herein is included within a chewing gum of any of the currently available forms, such that the chewing gum is retained in the oral cavity for dissolving the overall formula and enabling the CGA matrix of the formula to be bio-available for absorption through sublingual membranes.

Other preferred embodiments of the present invention include variations of the formula for use in a topical lotion to be applied to the skin of human subjects such that CGA may be absorbed through the skin. In alternative embodiments, rather than CGA, an alternative polyphenol phytochemical may be used, most notably ECGC. As will be evident to those of ordinary skill in the art, the formulation for such embodiments would follow the normal formulas as described previously herein but excludes flavoring elements and the like.

Still other embodiments may be in the form of other forms of the confection such as taffy or slow-dissolving candy, and other preferred embodiments include a powdered preparation following the formulas as described herein. In use, such a powder may be combined with coffee or other food products to enable delivery of CGA to human subjects through ingestion.

To fully understand the invention despite the more particular wording used in certain parts of these descriptions, it is important for the reader to presume that each part of this description is illustrative of more-general inventive concepts. Except to the extent limited by the prior art and prosecution, or to the extent expressly and unequivocally limited in a particular context, it should be understood that reference to any chemical entity should be construed as broadly as possible for purposes of these descriptions. Accordingly, even if the molecular formula and structure for a compound or group of compounds may be generally expressed in limited ways, it should be assumed that reference to such compound or group broadly encompasses any compound that substantially fulfils at least one of the corresponding definitions that may be used for that compound or group in the public domain.

For instance, this description often generally refers to CGA as a specific compound even though those of skill in the art would recognize that CGA has many variations in its formula and structure, such as (without limitation) variations of caffeoylquinic, feruloylquinic and dicaffeoylquinic acids, all of which should be understood as falling within the scope of CGA. Likewise, with any compound referenced herein, unless Applicant expressly and unequivocally clarifies that the reference should be more limited in a particular context, all references to that compound should be presumed to read as broadly as possible for purposes of construing the scope of the invention claimed.

In addition, to the extent permitted by the prior art and prosecution, any reference in the claims or elsewhere to a particular compound or group should also be understood as encompassing equivalents, including equivalents that would accomplish substantially the same function as the referenced compound or group. To that same extent, i.e., so long as there is not a requirement for a more limited interpretation in the prosecution history and/or based on an otherwise invalidating prior art reference, such equivalents should be presumed to include all homologues, isomers, lactones, metabolites, derivatives, fragments, variants, analogs, substitutes, alternatives and the like.

In all respects, it should also be understood that any particular embodiments, formulations and descriptions herein are to be regarded in an illustrative rather than a restrictive manner, and are not intended to limit the invention to the particular forms and examples disclosed. Rather, the invention includes all embodiments and methods within the scope and spirit of the invention as claimed, as the claims may be later added, amended, replaced or otherwise modified during the course of related prosecution. Any current, amended or added claims should be interpreted to embrace all further modifications, changes, rearrangements, substitutions, alternatives, design choices and embodiments that may be evident to those of skill in the art, whether now known or later discovered. In any case, all substantially equivalent systems, articles, and methods should be considered within the scope of the invention and, absent express indication otherwise, and all structural or functional equivalents are anticipated to remain within the spirit and scope of the inventive product, preparation and method.

We claim:

1. A CGA food supplement substantially as described in the foregoing description.

2. A CGA food supplement comprising:
CGA; and
other components common to food supplements.

3. A method of providing a food supplement comprising adding CGA to a food supplement to form a CGA food supplement.

4. The method of claim 3, wherein the food supplement is produced in the form of a chewing gum.
